(19) United States
(12) Patent Application Publication
(10) Pub. No.: US 2025/0133984 A1
(43) Pub. Date: May 1, 2025

(54) SOIL NITROGEN CONTENT SOFT MEASUREMENT METHOD BASED ON THE CONTROL SYSTEM FOR ON-DEMAND FERTILIZATION OF CORN

(71) Applicant: Jilin University, Changchun (CN)

(72) Inventors: Jiangtao QI, Changchun (CN); Junbo ZHOU, Changchun (CN); Weirong ZHANG, Changchun (CN); Fangfang GAO, Changchun (CN); Zhiyuan BAO, Changchun (CN); Chenchen DING, Changchun (CN); Yang WANG, Changchun (CN); Mingyang LV, Changchun (CN)

(73) Assignee: Jilin University, Changchun (CN)

(21) Appl. No.: 18/758,054

(22) Filed: Jun. 28, 2024

(30) Foreign Application Priority Data
Oct. 26, 2023 (CN) 202311393771 .7

(51) Int. Cl. A01C 21/00; G01N 33/24

(52) U.S. Cl. CPC A01C 21/007; G01N 33/245

(57) ABSTRACT

Disclosed are a maize on-demand fertilization control system and a soil nitrogen soft measurement method. Sensors transmit real-time data to a control unit, which calculates soil nitrogen content using an optimized BP neural network model. Based on maize's nitrogen demand, the system adjusts fertilization amounts using solenoid valves, achieving precise, cost-effective fertilization.

### CROSS-REFERENCE TO RELATED APPLICATIONS

This application claims priority to Chinese Patent Application No. 202311393771.7, filed on Oct. 26, 2023, the content of which is incorporated herein by reference in its entirety.

### TECHNICAL FIELD

The present application belongs to the field of intelligent agriculture equipment technology, and more specifically relates to a method comprising soil nitrogen content soft measurement based on the control system for on-demand fertilization of corn.

### BACKGROUND

Corn plays a crucial role in agricultural development in China. Being a high-efficiency C4 plant, corn requires various nutrients for growth, with nitrogen being the most essential. Adequate nitrogen is necessary for normal metabolic activities of corn plants. Additionally, corn is a high-yield and fertilization-tolerant crop, where the application of nitrogen fertilizer significantly affects its growth. However, precision fertilization techniques for corn currently suffer from issues such as low accuracy, lack of soil feedback information, and high costs. Therefore, there is an urgent need for a low-cost, high-precision corn fertilization device.

Soft measurement technology is a method of measuring variables that are difficult or temporarily impossible to measure using easily measurable variables. In the field of agricultural equipment, soft measurement techniques are often employed to measure unknown sensor data to save costs on expensive sensors or to measure difficult-to-measure variables. For instance, as disclosed in Chinese Patent Application Publication No. CN115952728A, some researchers have proposed a method for predicting the concentration of nitrite nitrogen in aquaculture water using an improved neural network-based soft measurement approach. This method establishes a mathematical model relating nitrite nitrogen concentration to other easily measurable water quality indicators, and then inputs the collected relevant water quality indicators into the developed improved neural network to indirectly measure the nitrite nitrogen concentration in aquaculture water.

The use of Back Propagation (BP) neural networks has become an important method for parameter soft measurement. In addressing the shortcomings of BP neural networks, some researchers have employed the Particle Swarm Optimization (PSO) algorithm to optimize BP neural networks. However, the PSO algorithm, while having the advantage of fast convergence, also suffers from drawbacks such as premature convergence and susceptibility to local optima, making it challenging to meet practical application requirements. Therefore, there is an urgent need to design an improved optimization algorithm to optimize BP neural networks and achieve high-precision soft measurement, thereby realizing feedback-based precision fertilization for corn.

### SUMMARY

The main purpose of the present application is to provide a rocker apparatus, aiming to improve the control accuracy of the rocker apparatus.

In response to the shortcomings of existing technologies, the present application provides a corn on-demand fertilization control system based on soil nitrogen content soft measurement. It utilizes the Optimize Adaptive Variation-Improved Inertial Weight-Whale Grey Wolf Optimizer-Sine Cosine Quantum Particle Swarm Optimization (OAV-IIW-WGWO-SCQPSO) algorithm to optimize the structure of the Back Propagation (BP) neural network and establish a soil nitrogen content soft measurement model, enabling real-time measurement of soil nitrogen content and precise control of corn fertilization quantities.

The present application relates to a control system for the on-demand fertilization of corn, the control system for on-demand fertilization of corn consists of a control unit, an electromagnetic valve assembly, a fertilization flow rate sensor assembly, a soil pH sensor, a soil moisture sensor, and a wireless data transmission unit. The electromagnetic valve assembly includes 12-24 electromagnetic valves, and the fertilization flow rate sensor assembly includes 12-24 fertilization flow rate sensors; the control unit is fixed to the upper end of the fertilization machine frame; each electromagnetic valve of the electromagnetic valve assembly is fixed between the nozzle and the output hole of the fertilization pipe; each fertilization flow rate sensor of the fertilization flow rate sensor assembly is fixed on the fertilization pipe and is located to the right of each output hole of the fertilization pipe; the nozzle is positioned directly above the corn seedlings; the soil pH sensor and the soil moisture sensor are connected to the wireless data transmission unit and are placed in the soil; the 12-24 electromagnetic valves of the electromagnetic valve assembly and the wireless data transmission unit are controlled by the control unit.

The present application relates to a method for soil nitrogen content soft measurement based on the control system for on-demand fertilization of corn, the method comprising the following steps:

S1. sampling the soil of corn with different fertilization amounts to obtain the fertilization amount data, measure soil pH value, soil moisture, total nitrogen content, available nitrogen, and hydrolyzable nitrogen, where the fertilization amount, soil pH value, and soil moisture are inputs to the model, and the total nitrogen content, available nitrogen, and hydrolyzable nitrogen are outputs, construct the training dataset for the soil nitrogen content soft measurement model.

S2. normalizing the data obtained in S1 using the following processing method:

$$y = ( {y}_{max} - {y}_{min} ) \times ( \frac{V - {x}_{min}}{{x}_{max} - {x}_{min}} ) + {y}_{min} .$$

Where: the y represents the normalized parameter data; the yₘₐₓ represents the maximum value of the expected normalization range; the yₘᵢₙ represents the minimum value of the expected normalization range; the xₘₐₓ represents the maximum value in each row of parameter data; the xₘᵢₙ represents the minimum value in each row of parameter data; and the V represents the actual parameter value.

S3. establishing a soil nitrogen content soft measurement model based on the Optimize Adaptive Variation-Improved Inertial Weight-Whale Grey Wolf Optimizer-Sine Cosine Quantum Particle Swarm Optimization algorithm to optimize the BP neural network and perform soil nitrogen content soft measurement.

S3.1. establishing a 3-layer BP neural network topology structure with 3 layers consisting of input layer, hidden layer, and output layer, the number of nodes in the input layer is 3, the number of nodes in the hidden layer is H, and the number of nodes in the output layer is 3, input the fertilization amount, soil pH value, and soil moisture data from the training dataset samples into the input layer, and corresponding expected outputs and actual outputs will be generated; initialize the number of nodes, weights, and thresholds for each layer of the BP neural network.

S3.2. optimizing the BP neural network using the Optimize Adaptive Variation-Improved Inertial Weight-Whale Grey Wolf Optimizer-Sine Cosine Quantum Particle Swarm Optimization algorithm, the optimization process includes the following steps:

S3.2.1. determining the particle dimension P_{d} in the Optimize Adaptive Variation-Improved Inertial Weight-Whale Grey Wolf Optimizer-Sine Cosine Quantum Particle Swarm Optimization algorithm, the calculation method is as follows: P_{d}=in+in·H+H+H·out+out.

Where: the in is the number of neurons in the input layer of the BP neural network; and the out is the number of neurons in the output layer of the BP neural network.

S3.2.2. determining the particle fitness function and calculate the fitness of each particle, the calculation method for the particle fitness function is:

$$Fi = \frac{{\sum }_{j = 1}^{N} {( {Y}_{j} - {y}_{j} )}^{2}}{N} .$$

Where: the Yⱼ represents the expected output of the j-th particle; and the yⱼ represents the actual output of the j-th particle.

S3.2.3. dividing the individuals α, β, and δ of the Optimize Adaptive Variation-Improved Inertial Weight-Whale Grey Wolf Optimizer-Sine Cosine Quantum Particle Swarm Optimization algorithm based on the size of their fitness.

S3.2.4. according to the Whale Grey Wolf Optimizer (WGWO) algorithm, update the particle positions under the guidance of the α, β, and δ individuals, the method of updating particle positions is as follows:

approaching the optimal solution: each particle approaches the optimal solution in the following manner:

$$\{ \begin{matrix}D = \vert C \cdot {x}_{p ( t )} - {x}_{t} \vert \\ {x}_{t + 1} = {x}_{p ( t )} - A \cdot D \\ A = 2 \cdot p \cdot {r}_{1} - p \\ C = 2 \cdot {r}_{2}\end{matrix} .$$

Where, the D is the Euclidean distance between the particle and the optimal solution; the x₍ₚ₍ₜ₎₎ is the position of the optimal solution; the xₜ is the particle position before starting the approach to the optimal solution process; the xₜ₊₁ is the particle position after finishing the approach to the optimal solution process; the A and C are variable coefficients; the p is the contraction factor, linearly decreasing from 2 to 0; the r₁ and r₂ are two distinct random numbers in the range [0,1].

Then search for the optimal solution, each particle searches for the optimal solution in the following manner:

$$\{ \begin{matrix}{D}_{q} = \vert {C}_{l} \cdot {x}_{l} - {x}_{t + 1} \vert \\ {x}_{l} = \{ \begin{matrix}{x}_{q} - {A}_{l} \cdot {D}_{q} & rand [ 0 , t ] \leq rand [ 0 , T ] , {r}_{3} < 0.5 \\ {x}_{q} + {D}_{q} {e}^{b \cdot R} \cdot cos ( 2 \cdot π \cdot R ) & rand [ 0 , t ] \leq rand [ 0 , T ] , {r}_{3} \geq 0.5 \\ {r}_{4} & rand [ 0 , t ] > rand [ 0 , T ]\end{matrix} \\ {x}_{t + 1}^{1} = ( 3 \cdot \overline{{ω}_{-}} \cdot {x}_{1} + 2 \cdot \overline{{ω}_{+}} \cdot {x}_{2} + \overline{{ω}_{+}} \cdot {x}_{3} ) / 6\end{matrix} .$$

Where: the q represents α, β, or δ; the D_{q} is the Euclidean distance between the particle and each of the α, β, or δ particles; the x₁ is the distance each particle moves towards the q particle; the x₁ is the distance each particle moves towards the α particle; the x₂ is the distance each particle moves towards the β particle; the x₃ is the distance each particle moves towards the δ particle; the b is the logarithmic spiral shape constant; the Ris a random number in the range [−1,1]; the r₃ and r₄ are random numbers in the range [0,1]; the rand[0,t] is a random number generated within the interval [0,t]; the rand[0,1] is a random number generated within the interval [0,T]; the xₜ₊₁¹ represents the particle position after searching for the optimal solution; the determination method for variable coefficients Aₗ and Cₗ is the same as that for A and C.

S3.2.5. calculating the particle fitness again.

S3.2.6. updating the particle velocity and perform secondary position updates based on the fitness of the particles: optimize the inertia weight in the Quantum Particle Swarm Optimization and introduce the optimized inertia weight value into the Whale Grey Wolf Optimizer (WGWO) algorithm, the updated formula for optimizing the inertia weight is as follows:

$$\{ \begin{matrix}\overline{{ω}_{-}} = \sqrt{{ω}_{max}^{2} - \frac{( {ω}_{max}^{2} - {ω}_{min}^{2} ) \cdot {t}^{2}}{{T}^{2}}} \\ \overline{{ω}_{+}} = \sqrt{{ω}_{max}^{2} - \frac{( {ω}_{max}^{2} - {ω}_{min}^{2} ) \cdot {( T - t )}^{2}}{{T}^{2}}}\end{matrix} .$$

Where: the ω̅₋̅ represents the decreasing inertia weight; the ω̅₊̅ represents the increasing inertia weight; the ωₘₐₓ represents the maximum inertia weight; the ωₘᵢₙ represents the minimum inertia weight; the T represents the number of iterations; the t represents the t-th iteration.

Incorporate the optimized inertia weight into the grey wolf hunting formula of the grey wolf algorithm, the particle velocity update formula of the particle swarm algorithm, and the particle position update formula of the particle swarm algorithm to iteratively update the particles.

S3.2.7. performing the third update on the particle position: when a random number generated within the interval [0,t] satisfies the mutation condition, i.e., when rand[0,t]>rand[0,T], based on the Optimize Adaptive Variation, perform the third update on the particle position, the update formula is as follows:

$${x}_{t + 1}^{3} = \{ \begin{matrix}{r}_{7} & rand [ 0 , t ] > rand [ 0 , T ] \\ {x}_{t + 1}^{3} & rand [ 0 , t ] \leq rand [ 0 , T ]\end{matrix} .$$

Where: the r₇ represents a random number generated within the interval [0,1]; the xₜ₊₁ represents the updated position of the particle.

S3.2.8. assigning the results of the third particle update to the weights and thresholds of the BP neural network; if the fitness value generated by the current iteration of the particle swarm update is less than that generated by the previous iteration, update the individual best and global best values; otherwise, proceed to the termination condition evaluation; if the number of iterations of the particle swarm update meets the termination condition, stop the update, and the BP neural network obtains the optimal weights and thresholds; otherwise, return to S3.2 to continue updating the weights and thresholds of the BP neural network.

S4. Inputting the real-time fertilization amount, soil pH value, and soil moisture data collected into the soil nitrogen content soft measurement model established in S3, and output the total nitrogen content, available nitrogen, and hydrolyzed nitrogen content of the soil.

Further, the selection rule for parameter H in S3.1 is as follows:

$$H = \{ \begin{matrix}[ min ( a , b , c ) , max ( a , b , c ) ] \\ a = 2 \cdot out + 1 \\ b = {log}_{2} ( in ) \\ c = \sqrt{in + out} + σ\end{matrix} .$$

Where: the σ is an integer between 1 and 10, and the a, b, c are positive integers; based on this, determine the range of values for H; train different BP neural networks with different values of H using training set data, obtain the corresponding network training accuracy errors, and finally select the H corresponding to the lowest network training accuracy error.

Further, the particle velocity update method in S3.2.6 is as follows:

$${v}_{t + 1} = \overline{{ω}_{-}} \cdot {v}_{t} + \overline{{ω}_{+}} \cdot {c}_{1} \cdot {rand}_{1} \cdot ( {p}_{best} - {x}_{t + 1}^{1} ) + \overline{{ω}_{-}} \cdot {c}_{2} \cdot {rand}_{2} \cdot ( {g}_{best} - {x}_{t + 1}^{1} ) .$$

Where: the vₜ₊₁ represents the updated particle velocity; the c₁ is the individual learning factor; the c₂ is the social learning factor; the rand₁ and the rand₂ are two distinct random numbers between [0,1]; the p_{best} is the current individual best solution of the particle; the g_{best} is the current global best solution of the particle.

Further, the particle position update method in S3.2.6 is as follows:

$${x}_{t + 1}^{2} = \{ \begin{matrix}\begin{matrix}\overline{{ω}_{+}} \cdot {v}_{t + 1} + \frac{{p}_{best ( t )} + {g}_{best ( t )}}{2} - \overline{{ω}_{-}} \cdot sin {r}_{5} \cdot \\ \vert {r}_{6} \cdot {\sum }_{i = 1}^{N} \frac{{p}_{ibest}}{N} - {x}_{t + 1}^{1} \vert \cdot ln ( \frac{1}{u} )\end{matrix} & u > 0.5 \\ \begin{matrix}\overline{{ω}_{+}} \cdot {v}_{t + 1} + \frac{{p}_{best ( t )} + {g}_{best ( t )}}{2} - \overline{{ω}_{-}} \cdot cos {r}_{5} \cdot \\ \vert {r}_{6} \cdot {\sum }_{i = 1}^{N} \frac{{p}_{ibest}}{N} - {x}_{t + 1}^{1} \vert \cdot ln ( \frac{1}{u} )\end{matrix} & u \leq 0.5\end{matrix} .$$

Where: the xₜ₊₁² represents the updated position of the particle from the swarm; the p_{ibest} represents the individual best position of the i-th particle in the swarm; the r₅ and the r₆ are random numbers between [0,1]; the N represents the total number of particles; the u represents a random number within the interval [0,1]; the p_{best} (t) represents the best position of the current particle up to now; the g_{best(t)} represents the best position of all particles up to now.

The present application offers the following beneficial effects.

Compared to existing variable fertilization technologies, such as those disclosed in Chinese Patent Publication No. CN114916298B, the on-demand fertilization device provided by this application utilizes soil pH sensors, soil moisture sensors, and fertilization flow sensors data, along with soil nitrogen content soft measurement methods, to measure real-time total nitrogen content, available nitrogen, and hydrolyzed nitrogen data in soil. This reduces the cost of required soil sensors. Additionally, it achieves feedback-based variable fertilization control based on real-time soil data. It adjusts the fertilization quantity based on soil nitrogen content (including total nitrogen content, available nitrogen, and hydrolyzed nitrogen data) and controls the opening of electromagnetic valves in the electromagnetic valve group. This significantly enhances the intelligence and precision of corn fertilization.

In the population algorithm-optimized BP neural network soft measurement model, first, the optimization efficiency of single-population algorithms is low and often fails to meet engineering practice requirements. Secondly, the inertia weight in the particle swarm algorithm of the population algorithm is fixed, resulting in low optimization accuracy of particles. Furthermore, the particle position updates in the particle swarm algorithm are continuous, with each update being heavily influenced by the previous update. Meanwhile, the population diversity in the later stages of the particle swarm algorithm significantly decreases, leading to a convergence of particles and severe limitations on the optimization space. If the grey wolf algorithm is used to optimize the particle swarm algorithm, it suffers from low optimization efficiency and accuracy. To address these issues, the present application proposes an optimization algorithm that improves the particle swarm algorithm using the grey wolf algorithm: two optimization inertia weights are proposed to change the individual leading role of α, β, and δ in the grey wolf algorithm at different times, thereby enhancing optimization efficiency. Additionally, the spiral position update method from the whale optimization algorithm is introduced to further improve the optimization accuracy of the grey wolf algorithm. Two optimization inertia weights are also introduced into the particle swarm algorithm velocity update formula, changing the weights of social learning and individual learning in different stages of the algorithm to enhance population diversity in the later stages. The particle positions updated through the grey wolf algorithm are incorporated into the particle swarm algorithm position update formula, enabling the algorithm to simultaneously take advantage of the strengths of both algorithms. Moreover, the position update formula of the particle swarm algorithm is optimized using quantum particle swarm update and cosine optimization strategies to expand the optimization space of the particle swarm algorithm. Finally, an improved adaptive mutation method, where mutation probability increases with the number of algorithm iterations, further optimizes the grey wolf algorithm and the particle swarm algorithm, enhancing the global search capabilities and optimization space of the particle swarm algorithm, thereby effectively improving the accuracy of the soil nitrogen content soft measurement model.

### BRIEF DESCRIPTION OF THE DRAWINGS

FIG. 1 is a schematic diagram of the structure of the corn on-demand fertilization control system.

FIG. 2 is a workflow diagram of the soil nitrogen content soft measurement model based on the OAV-IIW-WGWO-SCQPSO algorithm optimized BP neural network.

FIG. 3 is a convergence characteristic curve of the soil soft measurement model based on the PSO algorithm optimized BP neural network.

FIG. 4 is a convergence characteristic curve of the soil soft measurement model based on the OAV-IIW-WGWO-SCQPSO algorithm optimized BP neural network.

FIG. 5 is a particle fitness curve based on the PSO algorithm.

FIG. 6 is a Particle fitness curve based on the OAV-IIW-WGWO-SCQPSO algorithm.

### DETAILED DESCRIPTION OF THE EMBODIMENTS

The application is described below in conjunction with the attached drawings.

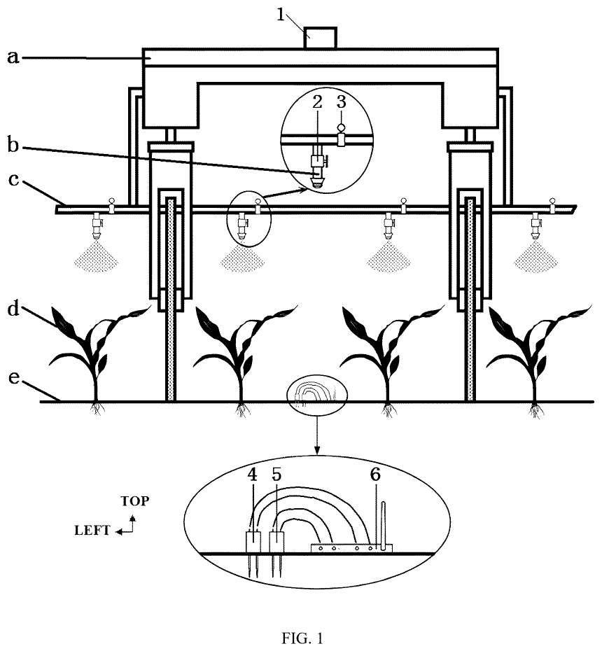

In FIG. 1, the application relates to a control system for on-demand fertilization of corn, the control system for on-demand fertilization of corn consists of a control unit 1, an electromagnetic valve assembly 2, a fertilization flow rate sensor assembly 3, a soil pH sensor 4, a soil moisture sensor 5, and a wireless data transmission unit 6, the electromagnetic valve assembly 2 includes 12-24 electromagnetic valves, and the fertilization flow rate sensor assembly 3 includes 12-24 fertilization flow rate sensors; the control unit 1 is fixed to the upper end of the fertilization machine frame a; each electromagnetic valve of the electromagnetic valve assembly 2 is fixed between the nozzle b and the output hole of the fertilization pipe c; each fertilization flow rate sensor of the fertilization flow rate sensor assembly 3 is fixed on the fertilization pipe c and is located to the right of each output hole of the fertilization pipe c; the nozzle b is positioned directly above the corn seedlings d; the soil pH sensor 4 and the soil moisture sensor 5 are connected to the wireless data transmission unit 6 and are placed in the soil e; the 12-24 electromagnetic valves of the electromagnetic valve assembly 2 and the wireless data transmission unit 6 are controlled by the control unit 1.

The fertilization flow rate sensor assembly 3, soil pH sensor 4, and soil moisture sensor 5 transmit real-time fertilization amount, soil pH value, and soil moisture data, respectively, to the control unit 1 through the wireless data transmission unit 6. The control unit 1 utilizes the real-time fertilization amount, soil pH, and soil moisture data, along with the soil nitrogen content soft measurement model, to calculate the real-time soil nitrogen content (including total nitrogen content, available nitrogen, and hydrolyzed nitrogen). Based on the nitrogen content requirements of corn in the soil, the control unit 1 determines whether the fertilization amount needs to be adjusted. If the current soil nitrogen content does not meet the nitrogen content requirements of corn, the control unit 1 adjusts the fertilization amount by controlling the opening of the electromagnetic valves in the electromagnetic valve assembly 2, thereby achieving on-demand fertilization control for corn.

The soil nitrogen content soft measurement method based on the corn on-demand fertilization control system of the present application utilizes the BP neural network model optimized by the OAV-IIW-WGWO-SCQPSO algorithm. Where, the OAV represents Optimize Adaptive Variation, the IIW represents Improved Inertial Weigh, the WGWO represents Whale Grey Wolf Optimizer, and the SCQPSO represents Sine Cosine Quantum Particle Swarm Optimization.

The specific process of the soil nitrogen content soft measurement method is as follows:

S1. sampling the soil of corn with different fertilization amounts to obtain the fertilization amount data, measure soil pH value, soil moisture, total nitrogen content, available nitrogen, and hydrolyzable nitrogen, where the fertilization amount, soil pH value, and soil moisture are inputs to the model, and the total nitrogen content, available nitrogen, and hydrolyzable nitrogen are outputs, construct the training dataset for the soil nitrogen content soft measurement model.

S2. normalizing the data obtained in S1 using the following processing method:

$$y = ( {y}_{max} - {y}_{min} ) \times ( \frac{V - {x}_{min}}{{x}_{max} - {x}_{min}} ) + {y}_{min} .$$

Where: the y represents the normalized parameter data; the yₘₐₓ represents the maximum value of the expected normalization range; the yₘᵢₙ represents the minimum value of the expected normalization range; the xₘₐₓ represents the maximum value in each row of parameter data; the xₘᵢₙ represents the minimum value in each row of parameter data; and the V represents the actual parameter value.

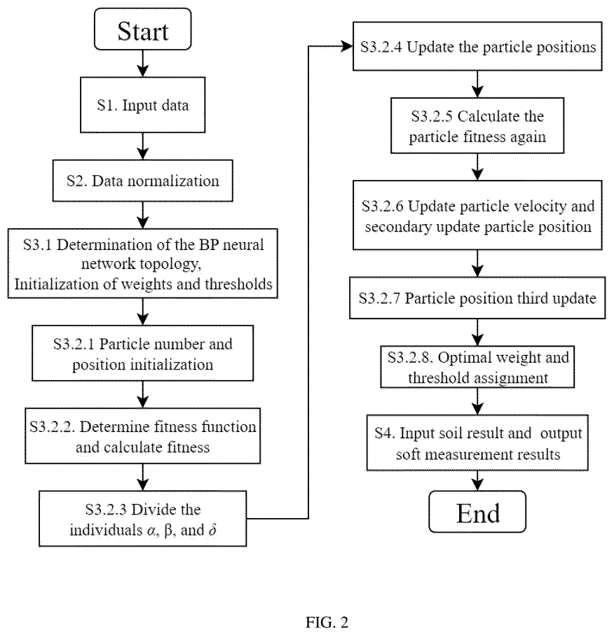

S3. Establishing a soil nitrogen content soft measurement model based on the OAV-IIW-WGWO-SCQPSO optimized BP neural network as shown in FIG. 2. The specific process is as follows:

S3.1. Establishing the BP neural network, establish a 3-layer BP neural network topology structure with 3 layers consisting of input layer, hidden layer, and output layer, the number of nodes in the input layer is 3, the number of nodes in the hidden layer is H, and the number of nodes in the output layer is 3, input the fertilization amount, soil pH value, and soil moisture data from the training dataset samples into the input layer, and corresponding expected outputs and actual outputs will be generated; initialize the number of nodes, weights, and thresholds for each layer of the BP neural network; the selection rule for parameter His as follows:

$$H = \{ \begin{matrix}[ min ( a , b , c ) , max ( a , b , c ) ] \\ a = 2 \cdot out + 1 \\ b = {log}_{2} ( in ) \\ c = \sqrt{in + out} + σ\end{matrix} .$$

Where: the in is the number of neurons in the input layer of the BP neural network; and the out is the number of neurons in the output layer of the BP neural network, the σ is an integer between 1 and 10, the a, b, and c are positive integers. Based on this, the range of H is determined to be [4,13]; train the BP neural network with different values of H using the training dataset, and obtain the corresponding network training accuracy errors, as shown in Table 1:

TABLE 1

| Value | Network Training Accuracy Error |
|---|---|
| 4 | 0.040 |
| 5 | 0.018 |
| 6 | 0.016 |
| 7 | 0.009 |
| 8 | 0.016 |
| 9 | 0.025 |
| 10 | 0.055 |
| 11 | 0.012 |
| 12 | 0.020 |
| 13 | 0.002 |

Select H with the lowest network training accuracy error, which is 13. Initialize the number of nodes, weights, and thresholds of the BP neural network: in=3, out=3, H=13; W₁=0.9, W₁ represents the weights from the input layer to the hidden layer; W₂=0.9, W₂ represents the weights from the hidden layer to the output layer; B₁=0.8, B₁ represents the thresholds from the input layer to the hidden layer; B₂=0.8, B₂ represents the thresholds from the hidden layer to the output layer.

S3.2. Optimizing the BP neural network using the OAV-IIW-WGWO-SCQPSO algorithm; use the GWO algorithm to improve the PSO algorithm and propose the OAV-IIW-WGWO-SCQPSO optimization algorithm to optimize the BP neural network. The IIW algorithm is used to propose two optimization inertia weight methods, changing the leading role of α, β, and δ individuals in different periods of the GWO algorithm to improve the optimization efficiency, and the spiral position update method of the whale optimization algorithm is introduced to propose the WGWO algorithm, further improving the optimization accuracy of the GWO algorithm. In the PSO algorithm, the IIW algorithm is introduced into the velocity update formula, changing the weights of social learning and individual learning in different periods of the PSO algorithm to enhance the diversity of particles in the later stage of the algorithm. The positions updated by the WGWO are introduced into the position update formula of the PSO algorithm, making the algorithm consider the advantages of both PSO and GWO algorithms. Additionally, the SCQPSO algorithm optimizes the position update formula of the PSO algorithm using the sine-cosine optimization (SC) strategy and the quantum particle swarm optimization (QPSO) position update, expanding the optimization space of the particle swarm algorithm. An OAV algorithm is proposed to increase the probability of mutation with the increase of the number of iterations, further optimizing the GWO and PSO algorithms, enhancing the global search capability and optimization space of the algorithm. The specific process is as follows:

S3.2.1. Determining the particle dimension Pa in the OAV-IIW-WGWO-SCQPSO algorithm.

The optimization object of the OAV-IIW-WGWO-SCQPSO algorithm is the weights and thresholds of the BP neural network. The dimension of the OAV-IIW-WGWO-SCQPSO algorithm particle is equal to the total number of parameters to be optimized, and the calculation formula is as follows: P_{d}=in+in·H+H+H·out+out.

S3.2.2. determining the particle fitness function and calculate the fitness of each particle, the calculation method for the particle fitness function is:

$$Fi = \frac{{\sum }_{j = 1}^{N} {( {Y}_{j} - {y}_{j} )}^{2}}{N} .$$

Where: the Fi represents the fitness function, the Yⱼ represents the expected output of the j-th particle; the yⱼ represents the actual output of the j-th particle, and the N represents the total number of particles.

S3.2.3. Dividing one α, β, and δ individual each based on the fitness size in the OAV-IIW-WGWO-SCQPSO algorithm, as shown below: Fi(α)<Fi(β)<Fi(δ)<Fi(other).

Where: the Fi(α) represents the fitness of the α individual in the population; the Fi(β) represents the fitness of the β individual in the population; the Fi(δ) represents the fitness of the δ individual in the population; the Fi(other) represents the fitness of the other individuals in the population.

S3.2.4. according to the Whale Grey Wolf Optimizer (WGWO) algorithm, update the particle positions under the guidance of the α, β, and δ individuals, the method of updating particle positions is as follows:

approaching the optimal solution: each particle approaches the optimal solution in the following manner:

$$\{ \begin{matrix}D = \vert C \cdot {x}_{p ( t )} - {x}_{t} \vert \\ {x}_{t + 1} = {x}_{p ( t )} - A \cdot D \\ A = 2 \cdot p \cdot {r}_{1} - p \\ C = 2 \cdot {r}_{2}\end{matrix} .$$

Where, the D is the Euclidean distance between the particle and the optimal solution; the x₍ₚ₍ₜ₎₎ is the position of the optimal solution; the xₜ is the particle position before starting the approach to the optimal solution process; the xₜ₊₁ is the particle position after finishing the approach to the optimal solution process; the A and C are variable coefficients; the p is the contraction factor, linearly decreasing from 2 to 0; the r₁ and r₂ are two distinct random numbers in the range [0,1];

then search for the optimal solution, each particle searches for the optimal solution in the following manner:

$$\{ \begin{matrix}{D}_{q} = \vert {C}_{l} \cdot {x}_{l} - {x}_{t + 1} \vert \\ {x}_{l} = \{ \begin{matrix}{x}_{q} - {A}_{l} \cdot {D}_{q} & rand [ 0 , t ] \leq rand [ 0 , T ] , {r}_{3} < 0.5 \\ {x}_{q} + {D}_{q} {e}^{b \cdot R} \cdot cos ( 2 \cdot π \cdot R ) & rand [ 0 , t ] \leq rand [ 0 , T ] , {r}_{3} \geq 0.5 \\ {r}_{4} & rand [ 0 , t ] > rand [ 0 , T ]\end{matrix} . \\ {x}_{t + 1}^{1} = ( 3 \cdot \overline{{ω}_{-}} \cdot {x}_{1} + 2 \cdot \overline{{ω}_{+}} \cdot {x}_{2} + \overline{{ω}_{+}} \cdot {x}_{3} ) / 6\end{matrix}$$

Where: the q represents α, β, or δ; the D_{q} is the Euclidean distance between the particle and each of the α, β, or δ particles; the xₗ is the distance each particle moves towards the q particle; the x₁ is the distance each particle moves towards the α particle; the x₂ is the distance each particle moves towards the β particle; the x₃ is the distance each particle moves towards the & particle; the b is the logarithmic spiral shape constant; the R is a random number in the range [−1,1]; the r₃ and r₄ are random numbers in the range [0,1]; the rand[0,t] is a random number generated within the interval [0,t]; the rand[0,T] is a random number generated within the interval [0,T]; the xₜ₊₁¹ represents the particle position after searching for the optimal solution; the determination method for variable coefficients Aₗ and Cₗ is the same as that for A and C.

S3.2.5. calculating the particle fitness again.

S3.2.6. updating the particle velocity and perform secondary position updates based on the fitness of the particles: optimize the inertia weight in the Quantum Particle Swarm Optimization and introduce the optimized inertia weight value into the Whale Grey Wolf Optimizer (WGWO) algorithm, the updated formula for optimizing the inertia weight is as follows:

$$\{ \begin{matrix}\overline{{ω}_{-}} = \sqrt{{ω}_{max}^{2} - \frac{( {ω}_{max}^{2} - {ω}_{min}^{2} ) \cdot {t}^{2}}{{T}^{2}}} \\ \overline{{ω}_{+}} = \sqrt{{ω}_{max}^{2} - \frac{( {ω}_{max}^{2} - {ω}_{min}^{2} ) \cdot {( T - t )}^{2}}{{T}^{2}}}\end{matrix} .$$

Where: the ω̅₋̅ represents the decreasing inertia weight; the ω̅₊̅ represents the increasing inertia weight; the ωₘₐₓ represents the maximum inertia weight; the ωₘᵢₙ represents the minimum inertia weight; the T represents the number of iterations; the t represents the t-th iteration.

Incorporating the optimized inertia weight into the grey wolf hunting formula of the GWO algorithm, the particle velocity update formula of the PSO algorithm, and the particle position update formula of the PSO algorithm to iteratively update the particles.

The particle velocity update method is as follows:

$${v}_{t + 1} = \overline{{ω}_{-}} \cdot {v}_{t} + \overline{{ω}_{+}} \cdot {c}_{1} \cdot {rand}_{1} \cdot ( {p}_{best} - {x}_{t + 1}^{1} ) + \overline{{ω}_{-}} \cdot {c}_{2} \cdot {rand}_{2} \cdot ( {g}_{best} - {x}_{t + 1}^{1} ) .$$

Where: the vₜ₊₁ represents the updated particle velocity; the c₁ is the individual learning factor; the c₂ is the social learning factor; the rand₁ and the rand₂ are two distinct random numbers between [0,1]; the p_{best} is the current individual best solution of the particle; the g_{best} is the current global best solution of the particle.

The second position update method of particles is as follows:

$${x}_{t + 1}^{2} = \{ \begin{matrix}\overline{{ω}_{+}} \cdot {v}_{t + 1} + \frac{{p}_{best ( t )} + {g}_{best ( t )}}{2} - \overline{{ω}_{-}} \cdot sin {r}_{5} \cdot \vert {r}_{6} \cdot {\sum }_{i = 1}^{N} \frac{{p}_{ibest}}{N} - {x}_{t + 1}^{1} \vert \cdot ln ( \frac{1}{u} ) & u > 0.5 \\ \overline{{ω}_{+}} \cdot {v}_{t + 1} + \frac{{p}_{best ( t )} + {g}_{best ( t )}}{2} - \overline{{ω}_{-}} \cdot cos {r}_{5} \cdot \vert {r}_{6} \cdot {\sum }_{i = 1}^{N} \frac{{p}_{ibest}}{N} - {x}_{t + 1}^{1} \vert \cdot ln ( \frac{1}{u} ) & u \leq 0.5\end{matrix} .$$

Where: the xₜ₊₁² represents the updated position of the particle from the swarm; the p_{ibest} represents the individual best position of the i-th particle in the swarm; the r₅ and the r₆ are random numbers between [0,1]; the N represents the total number of particles; the u represents a random number within the interval [0,1]; the p_{best} (t) represents the best position of the current particle up to now; the g_{best} (t) represents the best position of all particles up to now.

S3.2.7. performing the third update on the particle position: when a random number generated within the interval [0,t] satisfies the mutation condition, i.e., when rand[0,t]>rand[0,T], based on the Optimize Adaptive Variation, perform the third update on the particle position, the update formula is as follows:

$${x}_{t + 1}^{3} = \{ \begin{matrix}{r}_{7} & rand [ 0 , t ] > rand [ 0 , T ] \\ {x}_{t + 1}^{3} & rand [ 0 , t ] \leq rand [ 0 , T ]\end{matrix} .$$

Where: the r₇ represents a random number generated within the interval [0,1]; the xₜ₊₁³ represents the updated position of the particle.

S3.2.8. Every time the particle swarm updates its velocity and position (i.e., each time individual extreme value and global extreme value updates are performed), the weights and thresholds of the BP neural network are updated accordingly.

Compare the fitness values of the particles generated by the current particle swarm iteration updates with those generated by the previous generation's particle swarm iteration updates in real time. If the fitness value generated by the current particle swarm iteration updates is less than that generated by the previous generation's particle swarm iteration updates, continue with the individual extreme value and global extreme value updates. Otherwise, proceed to the termination condition judgment process.

Check whether the number of iterations of the particle swarm updates meets the preset termination conditions. If the number of iterations of the particle swarm updates meets the termination conditions, stop the updates, and the updates of the weights and thresholds of the BP neural network are also stopped, obtaining the optimal weights and thresholds. If the number of iterations of the particle swarm updates does not meet the termination conditions, return to S3.2 to continue updating the weights and thresholds of the BP neural network.

S4. Soil nitrogen content soft measurement, input the real-time fertilization amount, soil pH value, and soil moisture data collected into the soil nitrogen content soft measurement model established in S3, and output the total nitrogen content, available nitrogen, and hydrolyzed nitrogen content of the soil.

In traditional methods, the PSO algorithm is generally used to optimize the BP neural network to establish a soft measurement model. Compared to this, the performance in terms of particle fitness in this application is superior. Specific experimental data and analysis are as follows:

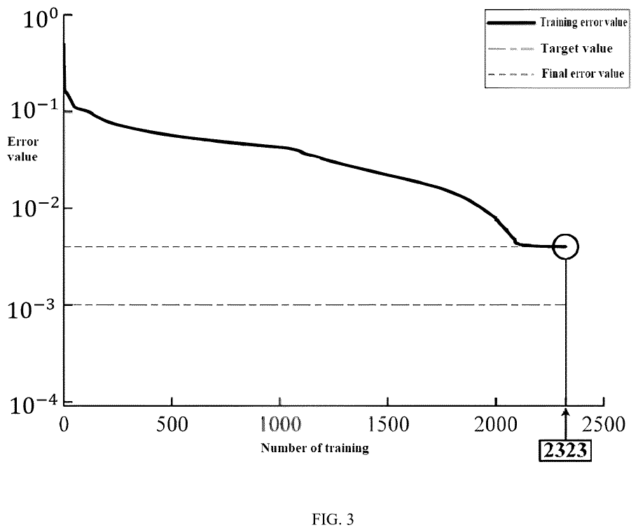

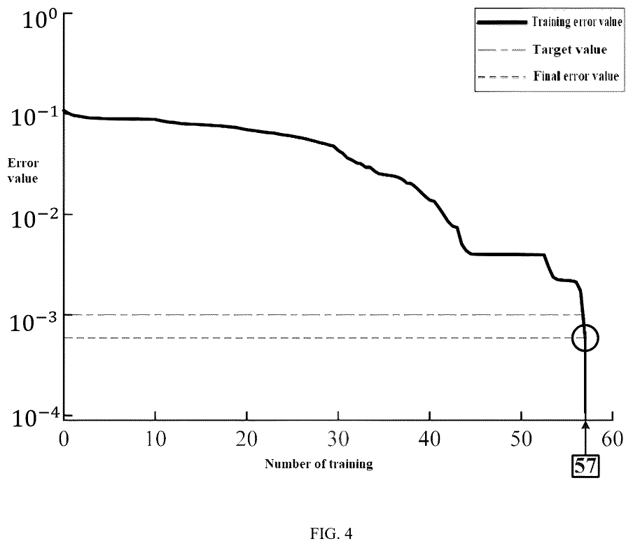

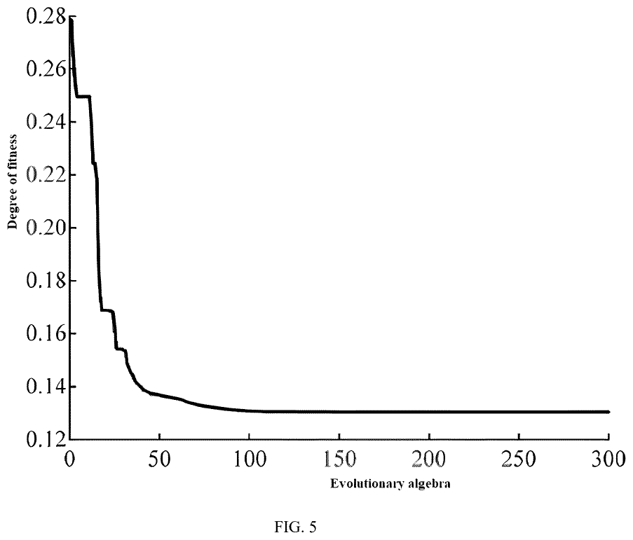

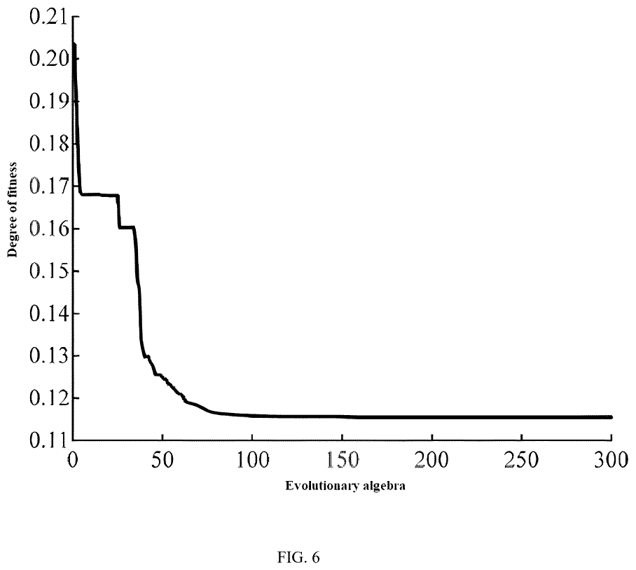

As shown in FIGS. 3 to 6, with a termination iteration number of 300 and a network training error target value of 0.001, when optimizing the BP neural network based on the PSO algorithm, the network training times are 2323, and the training error does not reach the target value. The final fitness of the particles in the particle swarm is 0.130. In contrast, when optimizing the BP neural network based on the OAV-IIW-WGWO-SCQPSO algorithm of this application, the network training times are 57, and the training error reaches the target value. The final fitness of the particles in the particle swarm is 0.116. Therefore, the soil nitrogen content soft measurement model established by this application exhibits superior performance.

What is claimed is:

1. A method for soil nitrogen content soft measurement based on a control system for on-demand fertilization of corn, the method comprising following steps:
S1: setting up the control system for on-demand fertilization of corn, wherein the control system for on-demand fertilization of corn comprises a control unit, an electromagnetic valve assembly, a fertilization flow rate sensor assembly, a soil pH sensor, a soil moisture sensor, and a wireless data transmission unit, the electromagnetic valve assembly comprises 12-24 electromagnetic valves, and the fertilization flow rate sensor assembly comprises 12-24 fertilization flow rate sensors; wherein the control unit is fixed to an upper end of a fertilization machine frame; each electromagnetic valve of an electromagnetic valve assembly is fixed between a nozzle and an output hole of a fertilization pipe; each fertilization flow rate sensor of the fertilization flow rate sensor assembly is fixed on the fertilization pipe and is located to the right of each output hole of the fertilization pipe; the nozzle is positioned directly above a corn seedlings; the soil pH sensor and the soil moisture sensor are connected to the wireless data transmission unit and are placed in the soil; the 12-24 electromagnetic valves of the electromagnetic valve assembly and the wireless data transmission unit are controlled by the control unit; sampling the soil of corn with different fertilization amounts to obtain the fertilization amount data, measuring soil pH value, soil moisture, total nitrogen content, available nitrogen, and hydrolyzable nitrogen, wherein the fertilization amount, soil pH value, and soil moisture are inputs to the model, and the total nitrogen content, available nitrogen, and hydrolyzable nitrogen are outputs for structuring the training dataset for the soil nitrogen content soft measurement model;
S2: normalizing the data obtained in S1 using the following processing method:
$$y = ( {y}_{max} - {y}_{min} ) \times ( \frac{V - {x}_{min}}{{x}_{max} - {x}_{min}} ) + {y}_{min} ;$$
wherein the y represents the normalized parameter data; the yₘₐₓ represents the maximum value of the expected normalization range; the yₘᵢₙ represents the minimum value of the expected normalization range; the xₘₐₓ represents the maximum value in each row of parameter data; the xₘᵢₙ represents the minimum value in each row of parameter data; and the V represents the actual parameter value;
S3: establishing a soil nitrogen content soft measurement model based on the Optimize Adaptive Variation-Improved Inertial Weight-Whale Grey Wolf Optimizer-Sine Cosine Quantum Particle Swarm Optimization (OAV-IIW-WGWO-SCQPSO) algorithm to optimize the BP neural network and perform soil nitrogen content soft measurement;
S3.1: establishing a 3-layer Back Propagation (BP) neural network topology structure with 3 layers consisting of input layer, hidden layer, and output layer, wherein the number of nodes in the input layer is 3, the number of nodes in the hidden layer is H, and the number of nodes in the output layer is 3, inputting the fertilization amount, soil pH value, and soil moisture data from the training dataset samples into the input layer, and corresponding expected outputs and actual outputs will be generated; initializing the number of nodes, weights, and thresholds for each layer of the BP neural network;
S3.2: optimizing the BP neural network using the Optimize Adaptive Variation-Improved Inertial Weight-Whale Grey Wolf Optimizer-Sine Cosine Quantum Particle Swarm Optimization algorithm, wherein the optimization process comprises the following steps:
S3.2.1: determining the particle dimension P_{d} in the Optimize Adaptive Variation-Improved Inertial Weight-Whale Grey Wolf Optimizer-Sine Cosine Quantum Particle Swarm Optimization algorithm, wherein the calculation method is as follows: P_{d}=in+in·H+H+H·out+out;
wherein the in is the number of neurons in the input layer of the BP neural network; and the out is the number of neurons in the output layer of the BP neural network;
S3.2.2: determining the particle fitness function and calculating the fitness of each particle, wherein the calculation method for the particle fitness function is:
$$Fi = \frac{{\sum }_{j = 1}^{N} {( {Y}_{j} - {y}_{j} )}^{2}}{N} ;$$
wherein the Yⱼ represents the expected output of the j-th particle; and the yⱼ represents the actual output of the j-th particle;
S3.2.3: dividing the individuals α, β, and δ of the Optimize Adaptive Variation-Improved Inertial Weight-Whale Grey Wolf Optimizer-Sine Cosine Quantum Particle Swarm Optimization algorithm based on the size of their fitness;
S3.2.4: according to the Whale Grey Wolf Optimizer (WGWO) algorithm, updating the particle positions under the guidance of the α, β, and δ individuals, wherein the method of updating particle positions is as follows:
approaching the optimal solution: each particle approaches the optimal solution in the following manner:
$$\{ \begin{matrix}D = \vert C \cdot {x}_{p ( t )} - {x}_{t} \vert \\ {x}_{t + 1} = {x}_{p ( t )} - A \cdot D \\ A = 2 \cdot p \cdot {r}_{1} - p \\ C = 2 \cdot {r}_{2}\end{matrix} ;$$
wherein the D is the Euclidean distance between the particle and the optimal solution; the x₍ₚ₍ₜ₎₎ is the position of the optimal solution; the xₜ is the particle position before starting the approach to the optimal solution process; the x₁₊₁ is the particle position after finishing the approach to the optimal solution process; the A and C are variable coefficients; the p is the contraction factor, linearly decreasing from 2 to 0; the r₁ and r₂ are two distinct random numbers in the range [0,1];
searching for the optimal solution, wherein each particle searches for the optimal solution in the following manner:
$$\{ \begin{matrix}{D}_{q} = \vert {C}_{l} \cdot {x}_{l} - {x}_{t + 1} \vert \\ {x}_{l} = \{ \begin{matrix}{x}_{q} - {A}_{l} \cdot {D}_{q} & rand [ 0 , t ] \leq rand [ 0 , T ] , {r}_{3} < 0.5 \\ {x}_{q} + {D}_{q} {e}^{b \cdot R} \cdot cos ( 2 \cdot π \cdot R ) & rand [ 0 , t ] \leq rand [ 0 , T ] , {r}_{3} \geq 0.5 \\ {r}_{4} & rand [ 0 , t ] > rand [ 0 , T ]\end{matrix} ; \\ {x}_{t + 1}^{1} = ( 3 \cdot \overline{{ω}_{-}} \cdot {x}_{1} + 2 \cdot \overline{{ω}_{+}} \cdot {x}_{2} + \overline{{ω}_{+}} \cdot {x}_{3} ) / 6\end{matrix}$$
wherein the q represents α, β, or δ; the D_{q} is the Euclidean distance between the particle and each of the α, β, or δ particles; the xₗ is the distance each particle moves towards the q particle; the x₁ is the distance each particle moves towards the α particle; the x₂ is the distance each particle moves towards the β particle; the x₃ is the distance each particle moves towards the & particle; the b is the logarithmic spiral shape constant; the R is a random number in the range [−1,1]; the r₃ and r₄ are random numbers in the range [0,1]; the rand[0,t] is a random number generated within the interval [0,t]; the rand[0,T] is a random number generated within the interval [0,T]; the xₜ₊₁¹ represents the particle position after searching for the optimal solution; the determination method for variable coefficients Aₗ and Cₗ is the same as that for A and C;
S3.2.5: calculating the particle fitness again;
S3.2.6: updating the particle velocity and perform secondary position updates based on the fitness of the particles: optimize the inertia weight in the Quantum Particle Swarm Optimization and introduce the optimized inertia weight value into the Whale Grey Wolf Optimizer (WGWO) algorithm, wherein the updated formula for optimizing the inertia weight is as follows:
$$\{ \begin{matrix}\overline{{ω}_{-}} = \sqrt{{ω}_{max}^{2} - \frac{( {ω}_{max}^{2} - {ω}_{min}^{2} ) \cdot {t}^{2}}{{T}^{2}}} \\ \overline{{ω}_{+}} = \sqrt{{ω}_{max}^{2} - \frac{( {ω}_{max}^{2} - {ω}_{min}^{2} ) \cdot {( T - t )}^{2}}{{T}^{2}}}\end{matrix} ;$$
wherein the ω̅₋̅ represents the decreasing inertia weight; the ω̅₊̅ represents the increasing inertia weight; the ωₘₐₓ represents the maximum inertia weight; the ωₘᵢₙ represents the minimum inertia weight; the T represents the number of iterations; the t represents the t-th iteration;
incorporating the optimized inertia weight into the grey wolf hunting formula of the grey wolf algorithm, wherein the particle velocity update formula of the particle swarm algorithm, and the particle position update formula of the particle swarm algorithm to iteratively update the particles;
S3.2.7: performing the third update on the particle position: when a random number generated within the interval [0,t] satisfies the mutation condition, i.e., when rand[0,t]>rand[0,T], based on the Optimize Adaptive Variation, perform the third update of particle position, the update formula is as follows:
$${x}_{t + 1}^{3} = \{ \begin{matrix}{r}_{7} & rand [ 0 , t ] > rand [ 0 , T ] \\ {x}_{t + 1}^{3} & rand [ 0 , t ] \leq rand [ 0 , T ]\end{matrix} ;$$
wherein the r₇ represents a random number generated within the interval [0,1]; the xₜ₊₁³ represents the updated position of the particle;
S3.2.8: assigning the results of the third particle update to the weights and thresholds of the BP neural network; if the fitness value generated by the current iteration of the particle swarm update is less than that generated by the previous iteration, update the individual best and global best values; otherwise, proceed to the termination condition evaluation; if the number of iterations of the particle swarm update meets the termination condition, stop the update, and the BP neural network obtains the optimal weights and thresholds; otherwise, return to S3.2 to continue updating the weights and thresholds of the BP neural network;
S4: inputting the real-time fertilization amount, soil pH value, and soil moisture data collected into the soil nitrogen content soft measurement model established in S3, and output the total nitrogen content, available nitrogen, and hydrolyzed nitrogen content of the soil.

2. The method of claim 1, wherein the selection rule for parameter H in S3.1 is as follows:
$$H = \{ \begin{matrix}[ min ( a , b , c ) , max ( a , b , c ) ] \\ a = 2 \cdot out + 1 \\ b = {log}_{2} ( in ) \\ c = \sqrt{in + out} + σ\end{matrix} ;$$
wherein the σ is an integer between 1 and 10, and the a, b, c are positive integers; based on this, determine the range of values for H; train different BP neural networks with different values of H using training set data, obtain the corresponding network training accuracy errors, and finally select the H corresponding to the lowest network training accuracy error.

3. The method of claim 1, wherein the particle velocity update method in S3.2.6 is as follows:
$${v}_{t + 1} = \overline{{ω}_{-}} \cdot {v}_{t} + \overline{{ω}_{+}} \cdot {c}_{1} \cdot {rand}_{1} \cdot ( {p}_{best} - {x}_{t + 1}^{1} ) + \overline{{ω}_{-}} \cdot {c}_{2} \cdot {rand}_{2} \cdot ( {g}_{best} - {x}_{t + 1}^{1} ) ;$$
wherein the vₜ₊₁ represents the updated particle velocity; the c₁ is the individual learning factor; the c₂ is the social learning factor; the rand and the rand₂ are two distinct random numbers between [0,1]; the p_{best} is the current individual best solution of the particle; the g_{best} is the current global best solution of the particle.

4. The method of claim 1, wherein the particle position update method in S3.2.6 is as follows:
$${x}_{t + 1}^{2} = \{ \begin{matrix}\overline{{ω}_{+}} \cdot {v}_{t + 1} + \frac{{p}_{best ( t )} + {g}_{best ( t )}}{2} - \overline{{ω}_{-}} \cdot sin {r}_{5} \cdot \vert {r}_{6} \cdot {\sum }_{i = 1}^{N} \frac{{p}_{ibest}}{N} - {x}_{t + 1}^{1} \vert \cdot ln ( \frac{1}{u} ) & u > 0.5 \\ \overline{{ω}_{+}} \cdot {v}_{t + 1} + \frac{{p}_{best ( t )} + {g}_{best ( t )}}{2} - \overline{{ω}_{-}} \cdot cos {r}_{5} \cdot \vert {r}_{6} \cdot {\sum }_{i = 1}^{N} \frac{{p}_{ibest}}{N} - {x}_{t + 1}^{1} \vert \cdot ln ( \frac{1}{u} ) & u \leq 0.5\end{matrix} ;$$
wherein the xₜ₊₁² represents the updated position of the particle from the swarm; the p_{ibest} represents the individual best position of the i-th particle in the swarm; the r₅ and the r₆ are random numbers between [0,1]; the N represents the total number of particles; the u represents a random number within the interval [0,1]; the p_{best(t)} represents the best position of the current particle up to now; the g_{best(t)} represents the best position of all particles up to now.
